(19) United States
(12) Patent Application Publication
(10) Pub. No.: US 2025/0134699 A1
(43) Pub. Date: May 1, 2025

(54) TONGUE SLEEVE DEVICE

(71) Applicant: Keith Ly, Pasadena, CA (US)

(72) Inventor: Keith Ly, Pasadena, CA (US)

(21) Appl. No.: 18/496,858

(22) Filed: Oct. 28, 2023

(51) Int. Cl. A61F 5/56

(52) U.S. Cl. CPC A61F 5/566

(57) ABSTRACT

According to an aspect of the present invention, there is provided a tongue sleeve allowing for the tongue to remain in a comfortable anatomical position with a lower frenulum slit, comprising: a first outer wall on a left side; a first outer base perpendicular to the first outer wall; a second outer wall on a right side; a second outer base perpendicular to the second outer wall; a connecting floor connecting the first outer base and the second outer base; a first inner wall perpendicular to the connecting floor on the left side; a second inner wall perpendicular to the connection floor on the left side; and a connecting ceiling connecting the first inner wall and the second inner wall.

### BACKGROUND

Snoring is often associated with a sleep disorder called obstructive sleep apnea. Sleep apnea is a serious sleep disorder characterized by repeated pauses in breathing during sleep. These pauses can last anywhere from a few seconds to several minutes and can occur hundreds of times a night.

There is evidence pointing to tongue muscle relaxation and sliding toward the back of throat causing obstructive sleep apnea. This is more pronounced in overweight individuals whose throat is excessively infiltrated with adipose tissue narrowing the airway and exaggerating the problem.

The causes of obstructive sleep apnea also include redundant soft tissues of the airway and relaxation of the muscles at the back of the throat. Another cause pertinent to the present invention is that the tongue is pulled backward (posteriorly) into the pharynx and blocks the upper airway in a sleeping person who is in the supine position. The tongue is pulled backward by the force of gravity.

The tongue is attached to the base of the lower jaw causing the tongue and the lower jaw to move forward and backward together. This anatomical fact is the basis of the ‘jaw thrust technique’ used by anesthesiologists and emergency medical personnel to reestablish a compromised upper airway in unconscious people. Moving the lower jaw forward will open the upper airway by moving the tongue forward.

The increased airflow that results from relieving the upper airway obstruction reduces airflow turbulence thereby reducing snoring and treats the obstructive sleep apnea.

Therapies used in the past to treat snoring and obstructive sleep apnea include straps, dental devices, continuous positive airway pressure (CPAP) and surgical procedures. Some dental devices which move the lower jaw forward, CPAP and surgery are recognized and frequently recommended modalities of treatment.

Straps presently being sold have proved ineffective in reducing snoring and obstructive sleep apnea. Simply closing the mouth of a sleeper who is in the supine position does not stop the force of gravity from pulling the lower jaw and the tongue backward (posteriorly) and obstructing the upper airway. The lower jaw slowly slides backward (posteriorly) unless the straps are fastened tightly enough to be uncomfortable and cut off circulation. Some straps actually pull the lower jaw backward aggravating upper airway obstruction.

Some dental devices which move the lower jaw forward, CPAP and surgery are recognized and frequently recommended modalities of treatment.

Sleep apnea can lead to a number of serious health problems, including:

Cardiovascular disease: Sleep apnea is a major risk factor for heart attack, stroke, and heart failure.

Type 2 diabetes: Sleep apnea can make it difficult to control blood sugar levels, increasing the risk of developing type 2 diabetes.

Obesity: Sleep apnea can make it difficult to lose weight and can contribute to weight gain.

Metabolic syndrome: Sleep apnea is often associated with metabolic syndrome, a cluster of conditions that includes high blood pressure, high blood sugar, and high cholesterol.

Other health problems: Sleep apnea can also be associated with a number of other health problems, including depression, anxiety, and cognitive impairment.

Sleep apnea is a major burden on the healthcare system. In the United States, it is estimated that sleep apnea costs the economy over $150 billion per year in healthcare costs and lost productivity.

The following are common symptoms of sleep apnea: snoring; daytime sleepiness; fatigue; morning headaches; difficulty concentrating; and waking up gasping for air.

There are a number of treatments for sleep apnea, including continuous positive airway pressure (CPAP) therapy, oral appliances including tongue retaining devices and surgery. However, many people with sleep apnea are undiagnosed or untreated.

Some solutions have been proposed in the prior art. For example, US20120234332A1 discloses an anti-snoring and anti-obstructive sleep apnea apparatus having a plate or metal frame tongue shelf splint to prevent the flaccid tongue falling back, a palate shelf splint projection to elevate the soft palate and prevents its vibration, incisors teeth receptacles or pockets sockets for jaw displacer to hold the mandible moved forwards, and to prevent it falling back held between the bite block, catheter-tubing to administer oxygen supplementation from the external source which reduces air turbulence and promotes nasal breathing, a submental suprahyoid muscle stimulator placed below the tongue, and above the mucous membrane. An injection port is provided to administer any therapeutic agents and local anesthetics to reduce the sensitivity of the tongue and oral cavity mucus membrane lining to the foreign objects. This device is used to deliver therapeutic agents to prevent and to treat halitosis and various diseases.

Nevertheless, prior art solutions are generally expensive and difficult to use. Effective, non-invasive and non-cumbersome solutions which alleviate snoring and obstructive sleep apnea by preventing the tongue of a person from sliding toward the back of the throat are desirable.

### SUMMARY OF INVENTION

Therefore, the present invention provides a relatively simple approach to hold the tongue to prevent its backward slide during sleep.

The invention is a sleeve that can be installed on the tongue to prevent snoring and apnea. The sleeve of the proposed invention restrains the tongue with molars anchors and bite blocks, thereby preventing snoring, teeth grinding and sleep apnea.

According to an aspect of the present invention, there is provided a tongue sleeve, comprising: a first outer wall on a left side; a first outer base perpendicular to the first outer wall; a second outer wall on a right side; a second outer base perpendicular to the second outer wall; a connecting floor connecting the first outer base and the second outer base; a first inner wall perpendicular to the connecting floor on the left side; a second inner wall perpendicular to the connection floor on the left side; and a connecting ceiling connecting the first inner wall and the second inner wall.

### BRIEF DESCRIPTION OF THE DRAWINGS

FIG. 1 illustrates a tongue sleeve device according to an embodiment of the present invention.

### DETAILED DESCRIPTION

The present invention is an inexpensive and comfortable tongue restraining sleeve. The tongue sleeve of the proposed invention allows for a closed mouth, stopping drying mouth and dry throat, while also preventing the airflow obstruction that causes a person to snore and experience mild to moderate sleep apnea.

The sleeve consists of two tracks on the left and right sides in which to hold the user's upper teeth and/or lower teeth. A central portion is designed to contain the user's tongue. A frenulum slit can account for the user's frenulum.

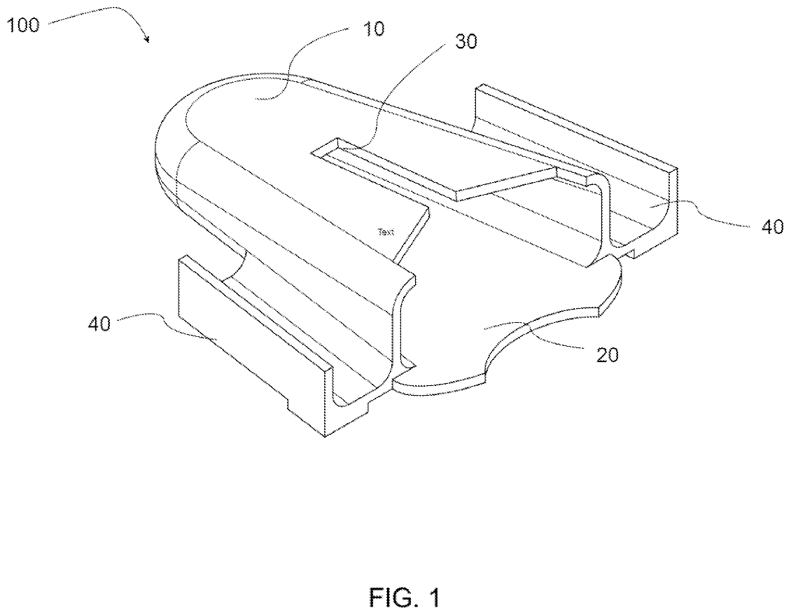

FIG. 1 illustrates tongue sleeve 100. Tongue sleeve ceiling 20 is designed to fit underneath the tongue of a user. Tongue sleeve floor 10 is designed to fit over the tongue of a user. Tongue sleeve floor has a slit for the frenulum of the user. Groove 30 can expand to allow the tongue sleeve to fit the shape and size of a user's tongue. Tracks 40 are positioned on the right and left side of the tongue sleeve. The tracks can be configured to accept the upper or lower teeth; in the view of FIG. 1, the tracks are configured to accept the lower teeth.

Instead of the tongue suction holding a user's tongue out of the mouth, the present invention is used inside the close mouth by a molar teeth anchor and tongue silicon sleeve with a frenulum slit. The proposed invention keeps the tongue inside the mouth and is thus more comfortable and there is no increase in salivation with tongue outside of the open mouth causing dryness of mucosa and teeth during the night.

The tongue will be held in its closed mouth anatomical and comfortable position without falling back during sleep obstructing the airway by using the tongue sleeve anchored to the premolars and molars. The sleeve covers the tongue with molars stabilizers and teeth grinding protectors.

The tongue sleeve can be made of a silicon material or any soft material. The tracks can be made of a hard silicon base. The molars have a teeth plate to help prevent teeth grinding with a harder silicon molars base and teeth grinding base.

The tongue sleeve has a molar base to hold the sleeve in place. The tongue sleeve encloses the tongue maximally on the upper side and minimally on the lower side with a frenulum slit to accommodate the natural tongue frenulum in an anatomical comfortable position.

This invention is superior to the tongue retaining devices in the market such as suction cups (pacifier like TRDs), which do not last on the tongue all night. Almost always they fall off in the middle of night because of air or saliva leaking into the suction cup. It also keeps the mouth somewhat open that results in dry mouth and irritation overnight. Suction cups cause severe strain on the tongue due to the pulling action and pull the tongue out of the mouth, keeping the tongue out of its anatomical position.

In contrast, the device of the present invention keeps the tongue in an anatomical natural comfortable position with the mouth closed while lying flat sleeping, while stabilizing the device to the molar teeth thus preventing the tongue from falling back into the throat obstructing the airway and causing snoring and sleep apnea.

The device of the present invention can be sold over the counter or can be sold by prescription. It can be sold in a stock shape or customized in shape for the patient's teeth and mouth based on the examination of a doctor or dentist.

The embodiments described above are given merely for example and for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A tongue sleeve, comprising:
a first outer wall on a left side;
a first outer base perpendicular to the first outer wall;
a second outer wall on a right side;
a second outer base perpendicular to the second outer wall;
a connecting floor connecting the first outer base and the second outer base;
a first inner wall perpendicular to the connecting floor on the left side;
a second inner wall perpendicular to the connection floor on the left side; and
a connecting ceiling connecting the first inner wall and the second inner wall.

2. The tongue sleeve of claim 1, further comprising a frenulum slit.

3. The tongue sleeve of claim 1, wherein the tongue sleeve is made of a material comprising silicone.

4. The tongue sleeve of claim 1, wherein the tongue sleeve is made of a material comprising rubber.

5. The tongue sleeve of claim 1, further comprising a groove can separate to allow the tongue sleeve to fit the shape and size of a user's tongue.

6. The tongue sleeve of claim 1, wherein the first outer base and second outer base are made from hard silicon.

7. The tongue sleeve of claim 1, wherein the first outer base and second outer base include a harder molar stabilizing portion on an outer edge.

8. The tongue sleeve of claim 1, wherein the first outer base and second outer base are customized based on the shape of the user's teeth

9. The tongue sleeve of claim 1, wherein the first outer base and second outer base are shaped to hold the upper teeth of a user.

10. The tongue sleeve of claim 1, wherein the first outer base and second outer base are shaped to hold the lower teeth of a user.
